(19) United States
(12) Patent Application Publication
(10) Pub. No.: US 2024/0229085 A1
(43) Pub. Date: Jul. 11, 2024

(54) Recombinant microorganism producing cannabigerolic acid and its derivatives thereof, and method for producing cannabigerolic acid and its derivatives thereof

(71) Applicant: NEOCANNBIO CO., LTD., Seoul (KR)

(72) Inventors: Jung Ho SONG, Seoul (KR); Jeong Kook KIM, Seoul (KR)

(73) Assignee: NEOCANNBIO CO., LTD., Seoul (KR)

(21) Appl. No.: 18/393,696

(22) Filed: Dec. 22, 2023

(30) Foreign Application Priority Data
Dec. 23, 2022 (KR) 10-2022-0183458
Dec. 20, 2023 (KR) 10-2023-0187559

(51) Int. Cl. C12P 7/42; C12N 1/20; C12N 9/10; C12N 15/70

(52) U.S. Cl. CPC C12P 7/42; C12N 1/20; C12N 9/1085; C12N 15/70; C12N 2501/999; C12Y 205/0101

(57) ABSTRACT

The disclosure relates to a polypeptide including a genetic mutation encoding prenyltransferase, a recombinant vector, a recombinant microorganism for producing cannabigerolic acid and derivatives thereof, and a method of producing cannabigerolic acid and derivatives thereof, wherein the recombinant microorganism for producing cannabigerolic acid and derivatives thereof is transformed with the recombinant vector have increased biosynthetic reactivity, enabling stable mass production of cannabigerolic acid and derivatives thereof, and the method of producing cannabigerolic acid and derivatives thereof may produce various cannabinoids with high productivity and yield at a low production cost.

### CROSS-REFERENCE TO RELATED APPLICATIONS

This application is based on and claims priority under 35 U.S.C. § 119 to Korean Patent Application No. 10-2022-0183458, filed on Dec. 23, 2022, and Korean Patent Application No. 10-2023-0187559, filed on Dec. 20, 2023 in the Korean Intellectual Property Office, the disclosures of which are incorporated by reference herein in their entirety.

### BACKGROUND

### 1. Technical Field

The disclosure relates to recombinant vectors including a genetic mutation encoding prenyltransferase, a recombinant microorganism producing cannabigerolic acid and derivatives thereof, and a method of producing cannabigerolic acid and derivatives thereof.

### 2. Description of the Related Art

Hemp (Cannabis sativa) has 480 or more natural constituents, 66 of which are classified as cannabinoids. Representative cannabinoids include tetrahydrocannabinol (THC), cannabidiol (CBD), cannabichromene (CBC), cannabigerol (CBG), cannabinol (CBN), cannabielsoin (CBE), cannabitriol (CBT), and cannabicyclol (CBL). Among these, tetrahydrocannabinol (THC) is a substance that causes addiction, while cannabidiol (CBD), unlike THC, does not have a psychoactive effect and is known to have an anti-anxiety effect that counteracts the psychoactive side effects of THC. CBD is used to treat pain from injuries, bumps, bruises, etc., inflammation, including arthritis, anxiety and panic disorders, nausea and vomiting, seizures and convulsive disorders, acne, and skin conditions, including rashes and eczema, etc. Cannabigerol (CBG) is a non-psychoactive cannabinoid that may be widely applied in the medical field. CBG is known to interact with CB1 and CB2 receptors in the endocannabinoid system to increase dopamine levels to regulate sleep, mood, and appetite, and to interfere with gamma-aminobutyric acid (GABA) receptor uptake in the brain and block serotonin receptors. CBG lowers anxiety, depression, intraocular pressure associated with glaucoma, etc., and blocks receptors that cause cancer cell growth, and even combats Staphylococcus aureus (MRSA). CBG is also effective in treating inflammatory bowel disease, neuronal degeneration, appetite stimulation, and bladder dysfunction.

Prenylation of natural compounds is known to add structural diversity, alter biological activity, and improve therapeutic potential, but prenylated compounds are often not abundant in nature or are difficult to isolate. Some prenylated natural products include a variety of bioactive molecules, including prenyl-flavanoids, prenyl-stilbenoids, cannabinoids, etc., which have been shown to be effective. Among these, cannabinoids such as tetrahydrocannabinol (THC) and cannabidiol (CBD) contained in hemp are known to have a very low content, making extraction difficult and expensive, making it difficult not only for medical use but also for research.

Cannabinoids are a class of bioactive plant-derived natural products that modulate the cannabinoid receptors (CB1 and CB2) of the human endocannabinoid system and are known to have anti-emetic, antispasmodic, analgesic, and antidepressant effects. In fact, cannabinoid therapy is understood to be FDA-approved for treating chemotherapy-induced nausea, multiple sclerosis (MS) spasms, and seizure associated with severe epilepsy. However, despite this therapeutic potential, the production of pharmaceutical-grade (>99%) cannabinoids still holds technical challenges to be solved. Cannabis plants such as marijuana and hemp include a variety of small amounts of cannabinoids and large amounts of tetrahydrocannabinolic acid (THCA) and cannabidiolic acid (CBDA). However, even cannabinoids such as CBDA and THCA, which are expressed at high levels, are difficult to isolate due to their structural similarity to contaminated cannabinoids and the variability in cannabinoid composition among plant species, which makes the isolation of rare cannabinoids even more challenging. Therefore, active research is underway to develop alternative methods for producing cannabinoids and cannabinoid analogs.

Under this background, the inventors of the disclosure have developed an improved variant enzyme with enhanced catalytic activity for cannabigerolic acid biosynthesis and developed a technology for introducing the enzyme into microorganisms for stable, large-scale production of the hemp-derived trace component cannabigerolic acid and derivatives thereof.

### SUMMARY OF THE INVENTION

Provided is a polypeptide including a prenyltransferase variant, wherein the prenyltransferase variant includes one or more amino acid substitutions in the amino acid sequence of SEQ ID NO: 2.

Provided is a polynucleotide encoding a polypeptide including the prenyltransferase variant.

Provided is a recombinant vector including a polynucleotide encoding a polypeptide including a prenyltransferase variant.

Provided is a recombinant microorganism for the production of cannabigerolic acid and derivative thereof with increased biosynthetic reactivity.

Provided is a method of producing cannabigerolic acid and derivative thereof.

The technical tasks to be accomplished in accordance with the technical ideas of the disclosure disclosed herein are not limited to the tasks to solve the challenges mentioned above, and other tasks not mentioned will be apparent to one of ordinary skill in the art from the following description.

Additional aspects will be set forth in part in the description which follows and, in part, will be apparent from the description, or may be learned by practice of the presented embodiments of the disclosure.

This is explained in detail as follows. Meanwhile, each description and embodiment disclosed herein may also be applied to each other description and embodiment. In other words, all combinations of the various elements disclosed herein fall within the scope of the application. Furthermore, the scope of the application is not to be considered limited by the specific descriptions set forth below.

The disclosure provides a polypeptide including a prenyltransferase variant, wherein the prenyltransferase variant may provide a polypeptide including one or more amino acid substitutions in the amino acid sequence of SEQ ID NO: 2.

The polypeptide may be a polypeptide including a genetic mutation encoding a prenyltransferase.

The prenyltransferase also called prenyl diphosphate synthase, is a type of enzyme that transfers a prenyl group to a receptor molecule and catalyzes the extension of the prenyl chain of prenyl diphosphate.

In the disclosure, the prenyltransferase may be an NphB enzyme that catalyzes the attachment of a 10-carbon geranyl group to an aromatic substrate.

The NphB enzyme may be a prenyltransferase derived from streptomyces, which may synthesize cannabigerolic acid using geranyl pyrophosphate and olivetolic acid as substrates, and introducing mutations in the active site of the enzyme may increase the reactivity of cannabigerolic acid and derivative thereof biosynthesis.

In the disclosure, the gene encoding prenyltransferase may include a known NphB gene sequence itself or a codon-optimized sequence tailored to the strain to be transformed. An example includes the nucleotide sequence of SEQ ID NO: 1, but is not limited thereto, considering mutations with biologically equivalent activity. In other words, a sequence that has ‘substantial identity’ with the sequence of the NphB gene or the nucleotide sequence of SEQ ID NO: 1 disclosed in the prior art may be included within the scope of the disclosure, and may refer to, for example, a sequence that exhibits 80% or more sequence homology, or 90% or more sequence homology.

As an example, a prenyltransferase gene of the disclosure may be a nucleotide sequence that has a sequence identity of within 81%, 82%, 83%, 84%, 85%, 86%, 87%, 88%, 89%, 90%, 91%, 92%, 93%, 94%, 95%, 96%, 97%, 98%, 99%, and 100% of the nucleotide sequence of SEQ ID NO: 1 within the range showing the biological activity of the NphB enzyme.

As an example, a prenyltransferase of the disclosure may be an amino acid sequence that has a sequence identity of within 81%, 82%, 83%, 84%, 85%, 86%, 87%, 88%, 89%, 90%, 91%, 92%, 93%, 94%, 95%, 96%, 97%, 98%, 99%, and 100% of the amino acid sequence of SEQ ID NO: 2 within the range showing the biological activity of the NphB enzyme.

The genetic mutation encoding the prenyltransferase may include a mutation in which at least one or more of the nucleotide sequences at the 139th, 145th, 637th, 640th, 646th, 694th, 805th, or 811st position of SEQ ID NO: 1 is substituted, which may be a substitution of the 47th, 49th, 213rd, 214th, 216th, 232nd, 269th, or 271st amino acid relative to the amino acid sequence of the prenyltransferase protein (SEQ ID NO: 2).

As an example, such genetic mutation encoding the prenyltransferase may include a mutation in which the nucleotide sequence at 139th position to 141st position, 145th position to 147th position, 637th position to 642nd position, 646th position to 648th position, 694th position to 696th position, 805th position to 807th position, 811st position to 813rd position in SEQ ID NO: 1 is substituted, and in the disclosure, the genetic mutation encoding the prenyltransferase may include at least one or more of the mutations described above.

According to an embodiment, the prenyltransferase variant may be represented by an amino acid sequence including one or more amino acid substitutions in the amino acid sequence of SEQ ID NO: 2, wherein the one or more amino acid substitutions may include substitutions of one or more amino acids selected from the group consisting of amino acids at the 47th, 49th, 213rd, 214th, 216th, 232nd, 269th, 271st, 286th and 288th position of SEQ ID NO: 2.

As an example, the prenyltransferase variant protein may refer to a protein expressed by an amino acid sequence including one or more of valine the 47th position in SEQ ID NO: 2 being substituted by a methionine, a leucine, an isoleucine, alanine, lysine, or arginine; valine at the 49th position is substituted by methionine, leucine, or isoleucine, alanine, lysine, or arginine; phenylalanine at the 213rd position is substituted by tyrosine, serine at the 214th position is substituted by alanine or threonine; tyrosine at the 216th position is substituted by phenylalanine, methionine, leucine, or isoleucine; alanine at the 232nd position is substituted for valine, methionine, leucine, or isoleucine; threonine at the 269th position is substituted for valine, methionine, leucine, or isoleucine; valine at the 271st position is substituted by phenylalanine: glycine at the 286th position is substituted by serine; and tyrosine at the 288th position of SEQ ID NO: 2 is substituted by alanine.

Preferably, the prenyltransferase variant protein may refer to a protein represented by an amino acid sequence including one or more of phenylalanine at the 213rd position of SEQ ID NO: 2 being substituted for tyrosine, and serine at the 214th position of SEQ ID NO: 2 being substituted for threonine.

As an example, the prenyltransferase variant protein may refer to a protein expressed by an amino acid sequence including a substitution of glycine for serine at the 286th position in SEQ ID NO: 2 and/or a substitution of tyrosine for alanine at the 288th position in SEQ ID NO: 2.

In the disclosure, a prenyltransferase protein including a genetic mutation may include at least one or more of the above-described mutations.

According to an embodiment, the prenyltransferase variant may be a protein (F213Y+G286S+Y288A) expressed by an amino acid sequence including and/or consisting of a phenylalanine at the 213rd position in SEQ ID NO: 2 substituted with a tyrosine, a substitution of glycine at the 286th position of SEQ ID NO: 2 for serine and a substitution of tyrosine at the 288th position of SEQ ID NO: 2 for alanine.

According to an embodiment, the prenyltransferase variant may be a protein (S214T+G286S+Y288A) expressed by an amino acid sequence including and/or consisting of a serine at the 214th position in SEQ ID NO: 2 substituted with a threonine, a substitution of glycine at the 286th position of SEQ ID NO: 2 for serine and a substitution of tyrosine at the 288th position of SEQ ID NO: 2 for alanine.

According to an embodiment, the prenyltransferase variant or polypeptide including the prenyltransferase variant may have increased enzymatic activity over the prenyltransferase wild type, specifically may have increased enzymatic activity to convert olivetolic acid and/or derivative thereof to cannabigerolic acid (CBGA) and/or derivative thereof.

According to an embodiment, a prenyltransferase variant (S214T+G286S+Y288A) represented as amino acid sequences including serine at the 214th position in SEQ ID NO: 2 substituted with threonine, glycine at the 286th position in SEQ ID NO: 2 substituted with serine, and tyrosine at the 288th position in SEQ ID NO: 2 substituted with alanine, or a polypeptide including the prenyltransferase variant is significantly superior in production efficiency of one or more of CBGA, CBGCA, CBGVA, and CBGA-C0 compared to other variants, and specifically, has excellent production efficiency of cannabigerolic acid or a derivative thereof including one or more of CBGA, CBGCA, and CBGVA.

In a polypeptide including a prenyltransferase variant of the disclosure, the N-terminus or C-terminus of the prenyltransferase variant may include a tag protein for purification of the protein, and is not limited to the type of tag protein suitable for the purpose of detection, isolation or purification of the protein. The tag protein may be, for example, but not limited to, a polyhistidine (his), FLAG, GST, or Myc protein.

As an example, the prenyltransferase variant of the disclosure may include a His tag at the N-terminus.

The polypeptide of the disclosure may further include an affinity tag, and is not limited to the type of tag protein as long as the tag protein may increase the probability of reaction between the prenyltransferase enzyme and the substrate. The affinity tag may be, for example, but is not limited to SUMO, TrxA, GSP, maltose-binding protein (MBP), Fh8, PelB, and MISTIC.

As an example, the polypeptide of the disclosure may include an MBP tag or a MISTIC tag, wherein the MISTIC tag may be linked to the C-terminus of the prenyltransferase variant.

The MBP tag may increase the reactivity of the biosynthesis of cannabigerolic acid and derivative thereof, by increasing enzyme solubility and inhibiting inclusion body production thereby increasing the probability of reaction between the enzyme and the substrate.

The MISTIC tag may increase the reactivity of the biosynthesis of cannabigerolic acid and derivative thereof, by expressing the enzyme on the surface and increasing the probability of reaction with the substrate.

Additionally, the disclosure may provide a polynucleotide encoding a polypeptide including a prenyltransferase variant.

The polynucleotide is preferred due to codon degeneracy or in consideration of codons preferred in organisms intended to express polypeptides including the prenyltransferase variant, various modifications may be made to the coding region as long as they do not change the amino acid sequence of the prenyltransferase variant expressed from the coding region, various modifications or modifications may be made in parts other than the coding region to the extent that they do not affect gene expression, and those skilled in the art will understand that such modified genes are also included within the scope of the disclosure. In other words, as long as the polynucleotide according to an aspect encodes a protein with equivalent activity, one or more nucleic acid bases may be mutated by substitution, deletion, insertion, or a combination thereof, and these are also included within the scope of the disclosure.

Additionally, the disclosure may provide a recombinant vector including a polynucleotide encoding a polypeptide including the prenyltransferase variant. As used herein, the term “recombinant vector” may be used as an expression vector for a target polypeptide that, when operably linked to a gene encoding the target polypeptide to be expressed, is capable of expressing the target polypeptide with high efficiency in an appropriate host cell, and the recombinant vector may be expressible in a host cell. The host cell may be a prokaryotic cell or a eukaryotic cell, and depending on the type of host cell, expression control sequences such as promoters, terminators, enhancers, etc., sequences for membrane targeting or secretion, etc. may be appropriately selected and combined in various ways according to the purpose.

Additionally, the disclosure may provide a recombinant microorganism for the production of cannabigerolic acid and derivative thereof.

Cannabigerolic acid (CBGA) is a type of cannabinoid present in the hemp plant and is known to be a precursor to the tetrahydrocannabinolic acid (THCA) cannabidiolic acid (CBDA), cannabichromenic acid (CBCA), and cannabigerol (CBG), and is known to promote early apoptosis as a natural inhibitor. Additionally, cannabigerolic acid exhibits antibacterial, anti-inflammatory, and pain-relieving effects, and has been reported to work with the endocannabinoid system to positively affect mood and appetite.

In the disclosure, cannabigerolic acid derivative refers to a compound that may be derived from cannabigerolic acid, and examples may include cannabigerorcinic acid (CBGCA), cannabigerovarinic acid (CBGVA), and CBGA-C0(3-Geranyl-2,4-hydroxybenzoic acid), but are not limited thereto.

The production of cannabigerolic acid and/or derivative thereof may refer to converting olivetolic acid and/or derivative thereof to cannabigerolic acid and/or derivative thereof. As an example, olivetolic acid derivatives may include, but are not limited to, orsellinic acid, varinolic acid, and OA-C0 (2,4-Dihydroxybenzoic acid).

The recombinant microorganism may be a microorganism transformed with the above-described recombinant vector, or may be a microorganism expressing the above-described prenyltransferase variant or a polypeptide including the same. The recombinant microorganism is characterized by an increased cannabigerolic acid biosynthesis reaction, and may be specifically E. coli, bacteria, yeast or fungi, preferably E. coli, but is not limited thereto.

In the disclosure, transformation may be performed by any method of transformation provided that the recombinant vector including a polynucleotide encoding a polypeptide including the prenyltransferase variant may be efficiently introduced into the host cell, for example, but not limited to, electroporation, protoplast transformation, Lit ion transformation, lithium acetate transformation, yeast cell wall dissolving enzyme transformation, etc.

Additionally, the disclosure may provide a method of producing cannabigerolic acid and derivative thereof.

The production method may include culturing a microorganism expressing a polypeptide including a prenyltransferase variant in a solution including a geranyl pyrophosphate and an olivetolic acid and/or derivative thereof.

The microorganism may be a microorganism that expresses a polypeptide including the above-described prenyltransferase variant, and may be a microorganism transformed by introducing a polynucleotide encoding polypeptide including the prenyltransferase variant or a recombinant vector including the polynucleotide.

According to an embodiment, the method of production includes 1) transforming a microorganism with a recombinant vector as described above, and 2) performing a whole-cell reaction with the transformed microorganism in a solution including geranyl pyrophosphate, olivetolic acid (OA), and/or derivatives thereof.

The terms cannabigerolic acid, derivative of cannabigerolic acid, recombinant vector, microorganism, and transformation in the disclosure are as described above.

As used herein, the term “whole-cell reaction” refers to a reaction in which a microorganism is utilized to synthesize or produce a compound by converting a substrate, as an example, in the disclosure, whole-cell E. coli may refer to producing cannabigerolic acid and/or cannabigerolic acid derivatives using olivetolic acid and/or derivatives thereof and geranyl pyrophosphate as substrates.

Specifically, in an embodiment, a recombinant vector including a mutation in the prenyltransferase gene was introduced into competent E. coli BL21(DE3), and then the transformed E. coli was selected with ampicillin. It was confirmed that the selected E. coli may produce (synthesize) cannabigerolic acid and various derivatives of cannabigerolic acid in high yield using olivetolic acid and geranyl pyrophosphate as substrates. The high yield productivity and the ability to produce various cannabigerolic acid derivatives are effects that may be achieved by optimizing the reactivity of the enzyme to the substrate through nucleotide sequence mutation at a specific position of the prenyltransferase gene and a specific tag protein.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A to 1D illustrate the relative activity of whole-cell responses of prenyltransferase (PT) mutants to cannabigerolic acid (CBGA) and CBGA derivatives (FIG. 1A.CBGA; FIG. 1B.CBGCA; FIG. 1C.CBGVA; FIG. 1D.CBGA-C0);

FIG. 2 shows the results of comparing the amounts of cannabigerolic acid (CBGA) produced (conversion rate) from synthetic GPP (geranyl pyrophosphate tetrabutyl ammonium salt) and reagent GPP (geranyl pyrophosphate lithium salt);

FIG. 3 shows the results of comparing the amount of cannabigerolic acid (CBGA) production (conversion rate) by buffer pH of the S214T mutant;

FIG. 4 shows the results of comparing the amount of cannabigerolic acid (CBGA) production (conversion rate) by concentration of synthetic GPP (geranyl pyrophosphate tetrabutyl ammonium salt);

FIG. 5 shows the results of comparing the amount of cannabigerolic acid (CBGA) production (conversion rate) by concentration of the S214T mutation;

A of FIGS. 6 and B of FIG. 6 show the results of comparing the amount of CBGA produced and the amount produced per hour when a control group and NphB mutations (S214T, Y288A, and G286S) of the disclosure were introduced;

FIG. 7A to FIG. 7D show the results of whole-cell reaction LC-MS analysis of cannabigerolic acid (CBGA) and CBGA derivatives;

A of FIGS. 8 and B of FIG. 8 show the results of comparing the amount of cannabigerolic acid (CBGA) production (conversion rate) by affinity tag type; and

FIG. 9A and FIG. 9B are diagrams illustrating vector maps including an affinity tag, wherein FIG. 9A shows the tag linked to the N-terminus of a gene and FIG. 9B shows the tag linked to the C-terminus of a gene.

### DETAILED DESCRIPTION OF THE EMBODIMENTS

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the configuration and effects of the disclosure will be described in more detail through examples. These examples are only for illustrating the disclosure, and the scope of the disclosure is not limited thereto.

### Example 1: Cellular Introduction of Mutant Prenyltransferase

### 1.1. Prenyltransferase Mutation Production

Streptomyces Sp. prenyltransferase (PT) gene was synthesized by Cosmogenetech (Seoul, Korea) after codon optimization, including an N-terminal His-tag. Prenyltransferase (PT) protein and its encoding sequence are listed in Table 1 below.

TABLE 1

| Sequence | Sequence (amino acid sequence or | SEQ ID |
|---|---|---|
| classification | nucleotide sequence) | NO |
| Prenyltransferase | atgagcgaagcagctgatgtcgaaagagtgtacgctgcgat | 1 |
| coding sequence | ggaagaggccgccggtcttttaggagtcgcttgcgccagag | |
| | ataagatctaccctctgttatctactttccaggacacattagtag | |
| | aggggggatctgttgtggtattctcgatggctagtggtcgccat | |
| | tcaactgaactggatttctctataagtgtacctacgagtcatgg | |
| | cgatccctatgccacggtagttgaaaaaggattatttccagct | |
| | acaggccatcccgttgatgacttactggctgatacacagaaa | |
| | cacttgccagtgagtatgtttgctatcgacggagaggttacag | |
| | gcggatttaagaagacgtatgcctttttcccaacggacaacat | |
| | gcctggcgtggctgaactttcagctattccttcgatgccaccgg | |
| | ccgtggccgagaatgcagaattgttcgctcgttatggcttaga | |
| | caaggtacaaatgaccagcatggattacaaaaagagaca | |
| | agttaatctttatttctctgaacttagcgctcaaacgttagaagct | |
| | gagagtgtattagctttagtgcgggagcttggcctgcacgttcc | |
| | aaacgaactgggattgaaattttgtaagcgttcattcagtgtat | |
| | atcctactcttaactgggaaacaggcaaaatcgaccggcttt | |
| | gctttgcagttatctcaaatgatccaactcttgtgccttcatctga | |
| | tgaaggcgacatagagaaattccataattatgccacaaagg | |
| | caccttatgcgtatgtcggagagaaacgcacccttgtttacgg | |
| | tttgaccttatccccgaaagaggagtattacaaattaggtgctt | |
| | attatcatataacagatgtgcaaagagggttgcttaaagcctt | |
| | cgacagccttgaagactga | |
| Prenyltransferase | MSEAADVERVYAAMEEAAGLLGVACARDKI | 2 |
| amino acid | YPLLSTFQDTLVEGGSVVVFSMASGRHSTE | |
| sequence | LDFSISVPTSHGDPYATVVEKGLFPATGHPV | |
| | DDLLADTQKHLPVSMFAIDGEVTGGFKKTY | |
| | AFFPTDNMPGVAELSAIPSMPPAVAENAEL | |
| | FARYGLDKVQMTSMDYKKRQVNLYFSELS | |
| | AQTLEAESVLALVRELGLHVPNELGLKFCK | |
| | RSFSVYPTLNWETGKIDRLCFAVISNDPTLV | |
| | PSSDEGDIEKFHNYATKAPYAYVGEKRTLV | |
| | YGLTLSPKEEYYKLGAYYHITDVQRGLLKAF | |
| | DSLED | |

In-Fusion cloning kit was purchased from Takara bio (Kyoto, Japan). Recipient E. coli DH5a and E. coli BL21 (DE3) were purchased from Invitrogen (Carlsbad, CA, USA). Olivetolic acid and geranyl pyrophosphate tetrabutyl ammonium salt were synthesized. Specifically, geranyl pyrophosphate tetrabutyl ammonium salt, 0.83 g (0.92 mmol) of tetrabutylammonium (tris) hydrogen pyrophosphate was added to a flame-dried 5 ml round bottom flask, filled with Ar₂, and 1.9 ml (0.24 M) of acetonitrile was injected into the mixture to prepare a white suspension. 0.1 g (0.46 mmol) of geranyl bromide was injected and reacted at room temperature overnight. After confirming that genaryl bromide disappeared by TLC, the reactant was synthesized by concentrating and removing the solvent to obtain a light yellow oil and solid mixture. Varinolic acid and orsellinic acid were purchased from Cayman Chemical (Michigan, USA). All other reagents were purchased from Sigma-Aldrich (St Louis, USA).

The mutation location of prenyltransferase were selected based on computer modeling, and each mutation was produced using a modified QuickChange™ site-directed mutagenesis (site-directed mutagenesis) method using pET22b(+)-PT as a template (Nucleic Acids Research. Vol. 32, No. 14, e115). Primer synthesis (Table 2) and sequence confirmation of the mutant genes were performed by Cosmogenetech (Seoul, Korea).

TABLE 2

| | | | SEQ ID |
|---|---|---|---|
| Primer classification | | Primer sequence (5′→3′ direction) | NO |
| V47L | Forward | GGA TCT ctt GTG GTA TTC TCG ATG | 3 |
| | | GCT | |
| | Reverse | TAC CAC aag AGA TCC CCC CTC TAC | 4 |
| | | TAA | |
| V47M | Forward | GGA TCT atg GTG GTA TTC TCG ATG | 5 |
| | | GCT | |
| | Reverse | TAC CAC cat AGA TCC CCC CTC TAC | 6 |
| | | TAA | |
| V47I | Forward | GGA TCT att GTG GTA TTC TCG ATG | 7 |
| | | GCT | |
| | Reverse | TAC CAC aat AGA TCC CCC CTC TAC | 8 |
| | | TAA | |
| V47A | Forward | GGA TCT gct GTG GTA TTC TCG ATG | 9 |
| | | GCT | |
| | Reverse | TAC CAC agc AGA TCC CCC CTC TAC | 10 |
| | | TAA | |
| V47K | Forward | GGA TCT aaa GTG GTA TTC TCG ATG | 11 |
| | | GCT | |
| | Reverse | TAC CAC ttt AGA TCC CCC CTC TAC | 12 |
| | | TAA | |
| V47R | Forward | GGA TCT cgt GTG GTA TTC TCG ATG | 13 |
| | | GCT | |
| | Reverse | TAC CAC acg AGA TCC CCC CTC TAC | 14 |
| | | TAA | |
| V49L | Forward | GTT GTG cta TTC TCG ATG GCT AGT | 15 |
| | | GGT | |
| | Reverse | CGA GAA tag CAC AAC AGA TCC CCC | 16 |
| | | CTC | |
| V49M | Forward | GTT GTG atg TTC TCG ATG GCT AGT | 17 |
| | | GGT | |
| | Reverse | CGA GAA cat CAC AAC AGA TCC CCC | 18 |
| | | CTC | |
| V49I | Forward | GTT GTG ata TTC TCG ATG GCT AGT | 19 |
| | | GGT | |
| | Reverse | CGA GAA tat CAC AAC AGA TCC CCC | 20 |
| | | CTC | |
| V49A | Forward | GTT GTG gca TTC TCG ATG GCT AGT | 21 |
| | | GGT | |
| | Reverse | CGA GAA tgc CAC AAC AGA TCC CCC | 22 |
| | | CTC | |
| V49K | Forward | GTT GTG aaa TTC TCG ATG GCT AGT | 23 |
| | | GGT | |
| | Reverse | CGA GAA ttt CAC AAC AGA TCC CCC | 24 |
| | | CTC | |
| V49R | Forward | GTT GTG cga TTC TCG ATG GCT AGT | 25 |
| | | GGT | |
| | Reverse | CGA GAA tcg CAC AAC AGA TCC CCC | 26 |
| | | CTC | |
| F213Y | Forward | CGT TCA tac AGT GTA TAT CCT ACT | 27 |
| | | CTT | |
| | Reverse | TAC ACT gta TGA ACG CTT ACA AAA | 28 |
| | | TTT | |
| S214T | Forward | TCA TTC act GTA TAT CCT ACT CTT | 29 |
| | | AAC | |
| | Reverse | ATA TAC agt GAA TGA ACG CTT ACA | 30 |
| | | AAA | |
| S214A | Forward | TCA TTC gct GTA TAT CCT ACT CTT | 31 |
| | | AAC | |
| | Reverse | ATA TAC agc GAA TGA ACG CTT ACA | 32 |
| | | AAA | |
| Y216F | Forward | AGT GTA ttt CCT ACT CTT AAC TGG | 33 |
| | | GAA | |
| | Reverse | AGT AGG aaa TAC ACT GAA TGA ACG | 34 |
| | | CTT | |
| Y216M | Forward | AGT GTA atg CCT ACT CTT AAC TGG | 35 |
| | | GAA | |
| | Reverse | AGT AGG cat TAC ACT GAA TGA ACG | 36 |
| | | CTT | |
| Y216L | Forward | AGT GTA ctt CCT ACT CTT AAC TGG | 37 |
| | | GAA | |
| | Reverse | AGT AGG aag TAC ACT GAA TGA ACG | 38 |
| | | CTT | |
| Y216I | Forward | AGT GTA att CCT ACT CTT AAC TGG | 39 |
| | | GAA | |
| | Reverse | AGT AGG aat TAC ACT GAA TGA ACG | 40 |
| | | CTT | |
| A232I | Forward | TGC TTT ata GTT ATC TCA AAT GAT | 41 |
| | | CCA | |
| | Reverse | GAT AAC tat AAA GCA AAG CCG GTC | 42 |
| | | GAT | |
| A232L | Forward | TGC TTT tta GTT ATC TCA AAT GAT | 43 |
| | | CCA | |
| | Reverse | GAT AAC taa AAA GCA AAG CCG GTC | 44 |
| | | GAT | |
| A232M | Forward | TGC TTT atg GTT ATC TCA AAT GAT | 45 |
| | | CCA | |
| | Reverse | GAT AAC cat AAA GCA AAG CCG GTC | 46 |
| | | GAT | |
| A232V | Forward | TGC TTT gta GTT ATC TCA AAT GAT | 47 |
| | | CCA | |
| | Reverse | GAT AAC tac AAA GCA AAG CCG GTC | 48 |
| | | GAT | |
| T269M | Forward | AAA CGC atg CTT GTT TAC GGT TTG | 49 |
| | | ACC | |
| | Reverse | AAC AAG cat GCG TTT CTC TCC GAC | 50 |
| | | ATA | |
| T269I | Forward | AAA CGC atc CTT GTT TAC GGT TTG | 51 |
| | | ACC | |
| | Reverse | AAC AAG gat GCG TTT CTC TCC GAC | 52 |
| | | ATA | |
| T269L | Forward | AAA CGC ctc CTT GTT TAC GGT TTG | 53 |
| | | ACC | |
| | Reverse | AAC AAG gag GCG TTT CTC TCC GAC | 54 |
| | | ATA | |
| T269V | Forward | AAA CGC gtc CTT GTT TAC GGT TTG | 55 |
| | | ACC | |
| | Reverse | AAC AAG gac GCG TTT CTC TCC GAC | 56 |
| | | ATA | |
| V271F | Forward | ACC CTT ttt TAC GGT TTG ACC TTA TCC | 57 |
| | Reverse | ACC GTA aaa AAG GGT GCG TTT CTC | 58 |
| | | TCC | |

*Codons at mutated positions are indicated in lowercase letters.

### 1.2. Intracellular Expression of Mutant Prenyltransferase

To express the proteins of each wild-type and mutant gene, chemical transformation was performed with competent E. coli BL21 (DE3), and then selected from LB (lysogeny broth)-agar plates including 100 μg/ml of ampicillin. The selected transformed BL21 cells were inoculated into 3 ml of liquid LB medium including 100 μg/ml of ampicillin and cultured at 37° C. and 200 rpm for 16 hours. Afterwards, the cells were secondarily inoculated with 50 ml of LB-ampicillin medium and cultured to an OD600 of 0.5, and then protein expression was induced with 0.8 mM isopropyl b-D-1-thiogalactopyranoside (IPTG) at 20° C. for 20 hours. After 20 hours, the cells were harvested and centrifuged (3000 rpm, 4° C.) to obtain a cell pellet.

### Example 2: Whole-Cell Reaction for Conversion of Cannabigerolic Acid and Derivative Thereof by Mutant Prenyltransferase

### 2.1. Conversion to Cannabigerolic Acid and Derivative Thereof

### (1) Experimental Method

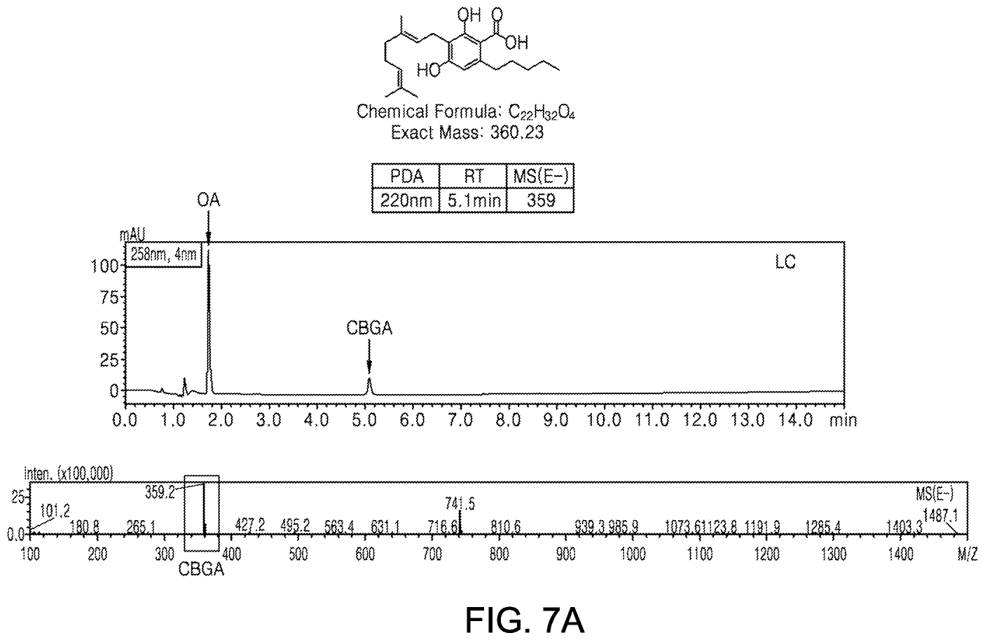

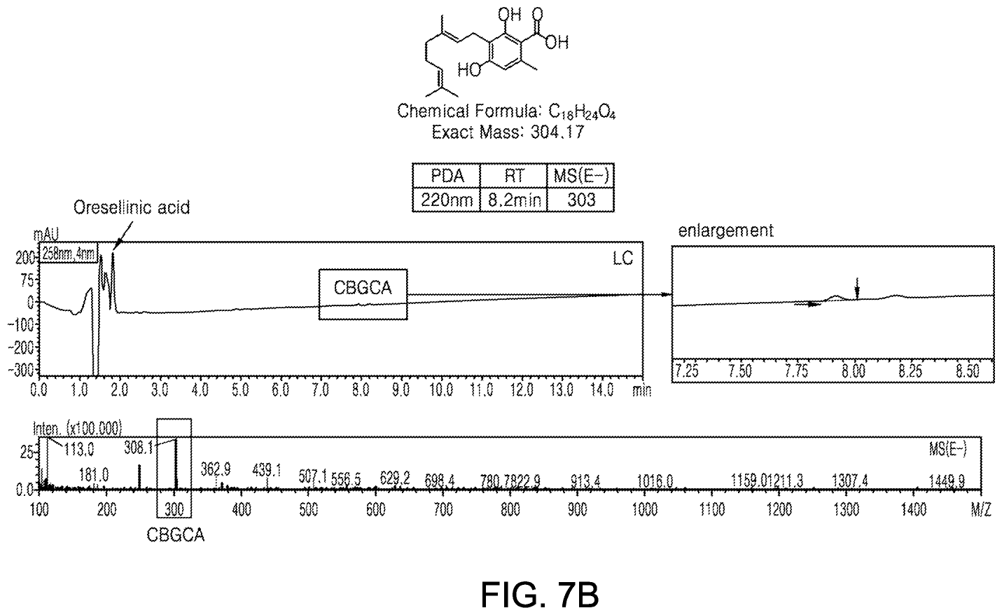

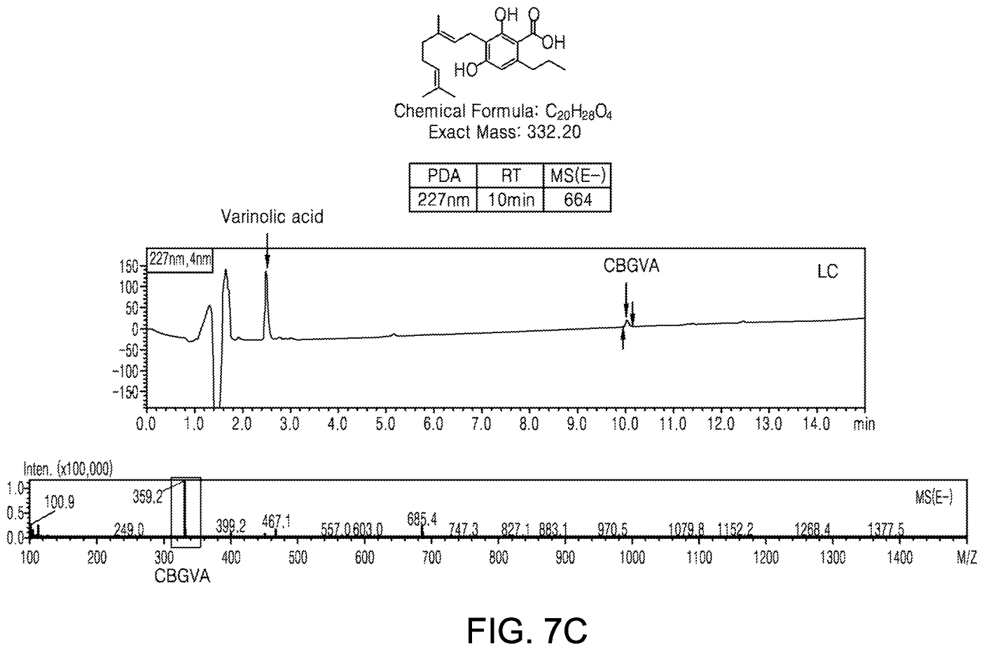

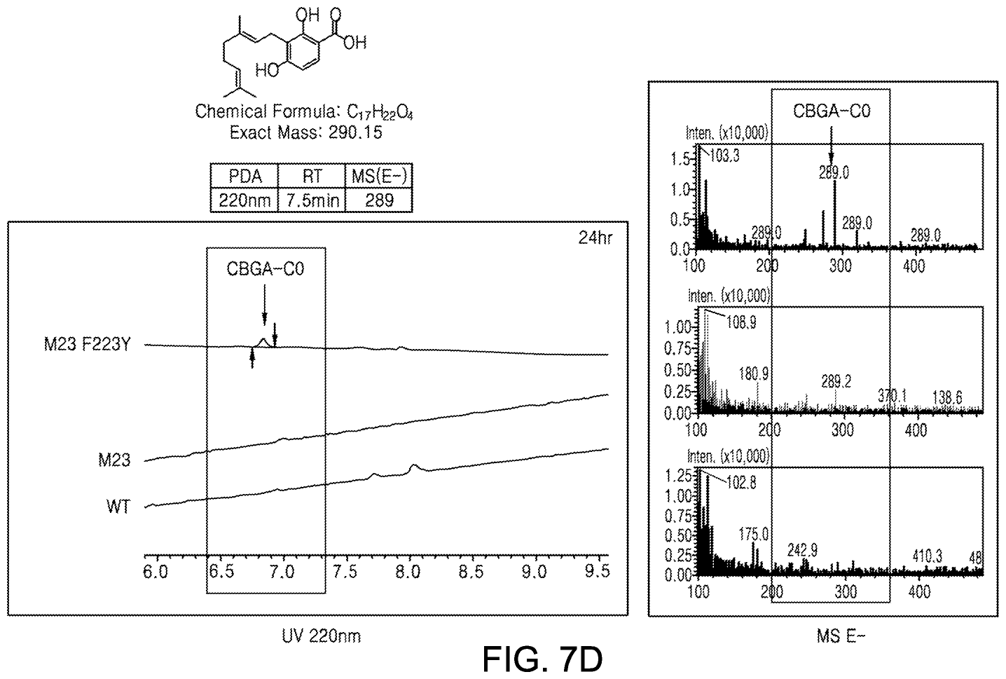

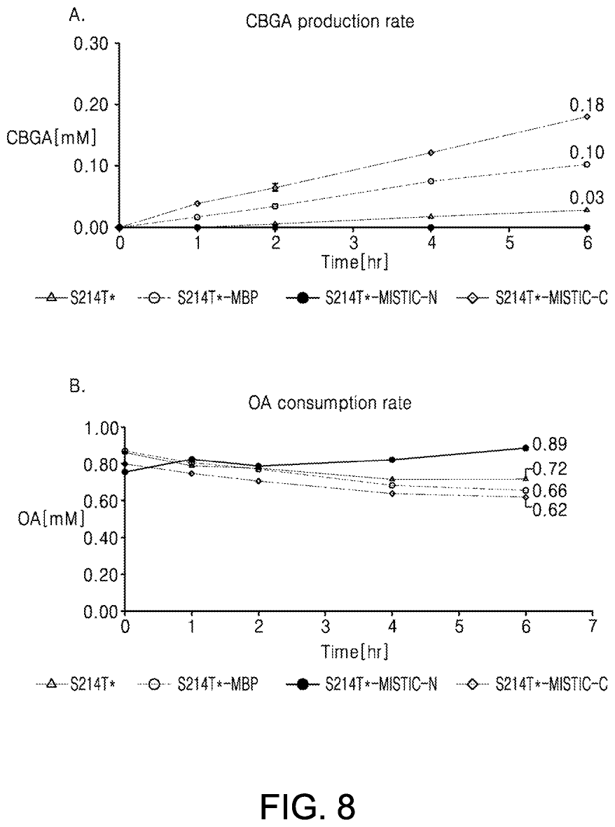

The conversion of olivetolic acid and derivative thereof to cannabigerolic acid and derivative thereof was carried out for 24 hours in 50 mM Tris-HCl buffer (pH 8.0) added with 0.9 gCDW/L cells, 1 mM olivetolic acid, 1 mM geranyl pyrophosphate lithium salt, and 5 mM magnesium chloride. After obtaining 100 ul samples at set time intervals, cells were inactivated by adding 900 ul of methanol, filtered with a 0.2 M PVDF filter (GE Healthcare, Pittsburgh, USA), and LC-MS analysis was performed (FIG. 7).

UPLC analysis was performed using an electrospray ionization-mass spectrometry (ESI-MS) Shimadzu LCMS-2020 system (Shimadzu, Kyoto, Japan) consisting of a solvent degassing unit (DGU-20A), binary pump (LC-30AD), autosampler (SIL-30AC), system controller unit (CBM-20A), photodiode array detector (SPD-M20A), and column oven unit (CTO-20AC) (analytical conditions: Table 3). Olivetolic acid and cannabigerolic acid were detected at each 1.7 minutes and 5.1 minutes at 258 nm and 220 nm PDA detectors, respectively, and [M-H]-type molecular ion peaks were confirmed at m/z 223.1 and m/z 359.2. Additionally, varinolic acid and CBGVA were detected at each 2.6 minutes and 10.1 minutes at 261 nm and 227 nm, and [M-H]-type molecular ion peaks were confirmed at m/z 195.2 and m/z 331.1. Orsellinic acid and CBGCA were detected at 261 nm and 220 nm at 1.9 minutes and 8.2 minutes, respectively, and [M-H]-type molecular ion peaks were confirmed at m/z 167.2 and m/z 303.1. OA-C0 and CBGA-C0 were detected at each 261 nm and 220 nm at 1.9 minutes and 7.5 minutes, and [M-H]-type molecular ion peaks were confirmed at m/z 153.1 and m/z 289.0 (FIG. 7).

TABLE 3

| | Item | Analysis conditions | | | | | |
|---|---|---|---|---|---|---|---|
| MS | Interface | ESI | | | | | |
| | DL tempera- | 250° C. | | | | | |
| | ture | | | | | | |
| | Heat Block | 200° C. | | | | | |
| | temperature | | | | | | |
| | PG Vacuum | 1.1e⁺⁰⁰² Pa | | | | | |
| | IG Vacuum | 3.5e⁻⁰⁰⁴ Pa | | | | | |
| | Nebulizing | 1.5 L/min | | | | | |
| | gas flow | | | | | | |
| | Dry gas flow | 15.0 L/min | | | | | |
| | Detector | 1.40 kV | | | | | |
| | voltage | | | | | | |
| LC | Column | Phenomenex Luna omega polar C18 100 | | | | | |
| | | (150 mm × 2.1 mm, 1.6 um) | | | | | |
| | Mobile | A) 0.1% formic acid in Water | | | | | |
| | phase | B) 0.1% formic acid in Acetonitrile | | | | | |
| | Flow Rate | 0.3 ml/min | | | | | |
| | Injection | 3 UI | | | | | |
| | amount | | | | | | |
| | Gradient | Time | | | Time | | |
| | condition | (minutes) | A(%) | B(%) | (minutes) | A(%) | B(%) |
| | | 0 | 30 | 70 | 0 | 50 | 50 |
| | | 11.5 | 0 | 100 | 11.5 | 20 | 80 |
| | | 13 | 0 | 100 | 13 | 20 | 80 |
| | | 13.5 | 30 | 70 | 13.5 | 50 | 50 |
| | | 15 | 30 | 70 | 15 | 50 | 50 |

| | | | |
|---|---|---|---|
| 18 | End | 18 | End |

| | |
|---|---|
| CBGA | CBGA derivative |

### (2) Experiment Results

The activity of NphB mutants (variant), a prenyltransferase selected based on computer modeling, toward cannabigerolic acid (CBGA) and CBGA derivatives was measured by whole-cell reaction, and based on the mutant (Y288A+G286S) in which a mutation was introduced at a specific position, the activity of the mutant was set at 100% and the relative response activities of mutants in which additional mutations were introduced were compared.

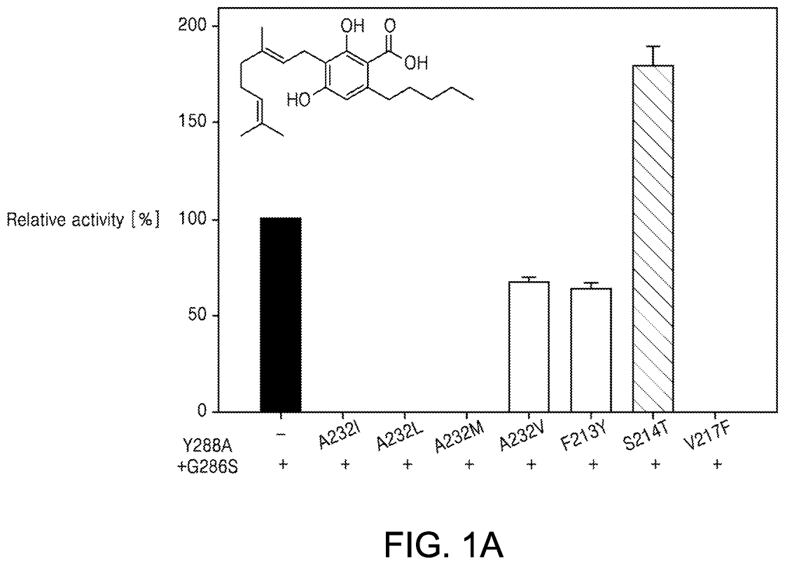

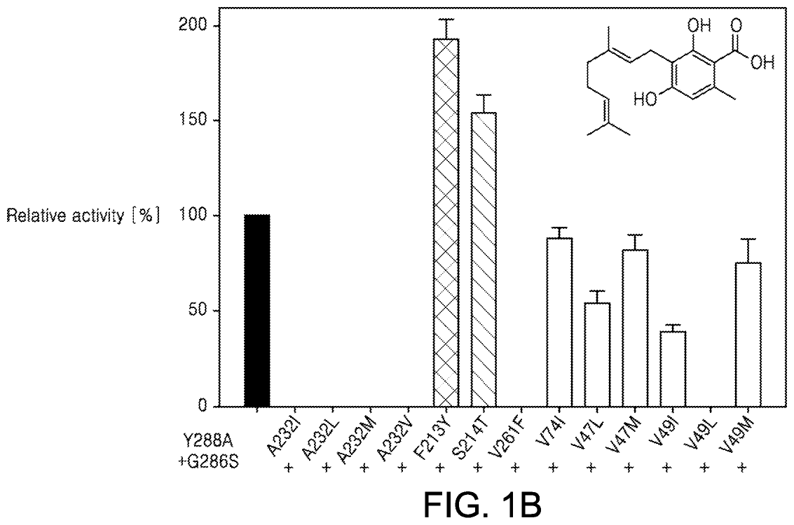

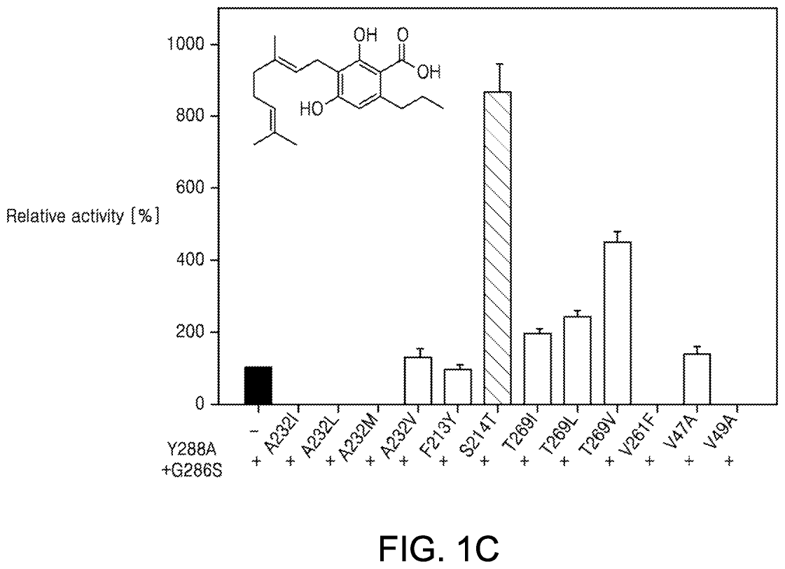

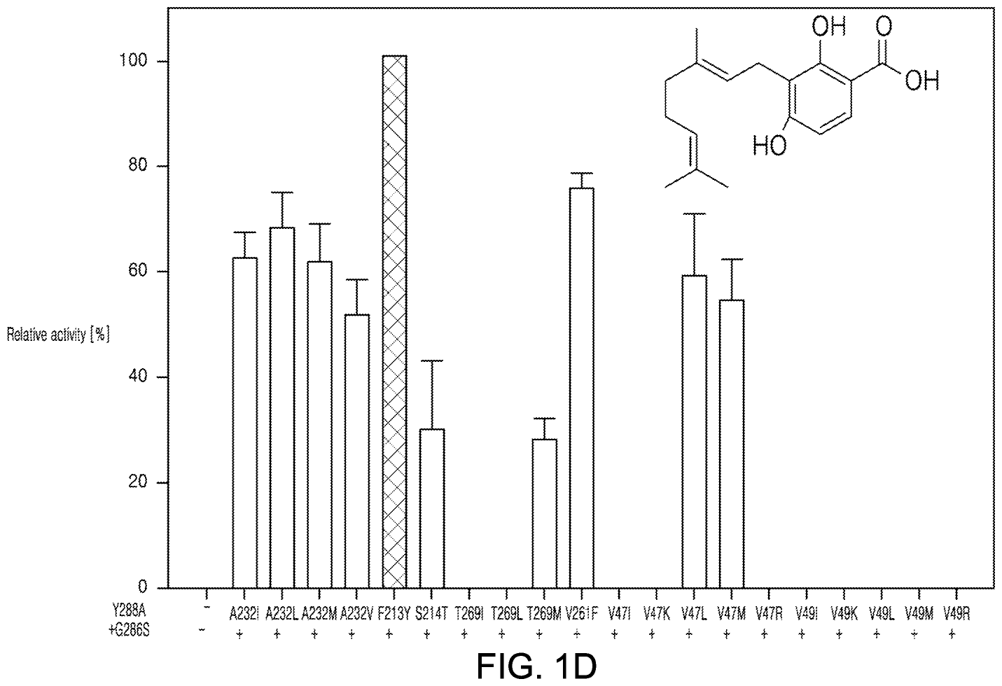

As a result, in the case of CBGA, the S214T mutant showed the highest activity at 180%, while the A232V and F213Y mutants showed levels of 67% and 64%, respectively (FIG. 1A).

In the case of CBGCA, the F213Y mutant showed the highest activity at 193%, and S214T had the second highest activity at 154%. The activities of the V471, V47L, V47M, V491, and V49M mutants were 88%, 54%, 82%, 39%, and 76%, respectively, which was lower than that of the reference mutant (Y288A+G286S) (FIG. 1B).

CBGVA showed the highest activity of the S214T mutant at 868%, and the activity of the A232V, F213Y, T2691, T269L, T269V, V47A, and V49A mutants were each 131%, 96%, 197%, 243%, 451%, and 140%, which were higher or similar to that of the reference mutant (Y288A+G286S) (FIG. 1C).

Notably, CBGA-C0 did not measure the activity of the reference mutant (Y288A+G286S), so the F213Y mutant, which had the highest activity, was set as 100%. As a result, A2321, A232L, A232M, and A232V were 62%, 68%, 62%, and 52%, respectively, S214T was 30%, T269M was 28%, V261F was 75%, and V47L and V47M were 59% and 54%, respectively (FIG. 1D).

As a result of screening, the mutant with additional introduction of S214T or F213Y showed higher activity than the reference mutant (Y288A+G286S) for most substrates. In the case of S214T and F213Y, a mutation was introduced into a residue common to the four substrates (olivetolic acid, orsellinic acid, varinolic acid and OA-C0(2,4-Dihydroxybenzoic acid)), and the mutation introduced into the residue common to the four substrates binds with the OH group at the 2nd position of the benzene ring to form a stable bond, resulting in the production of CBGA, CBGCA, CBGVA, CBGA-C0 as a product from each substrate.

### 2.2. Comparison of Cannabigerolic Acid Biosynthesis Efficiency of Synthetic GPP and Reagent GPP

The whole-cell reaction to compare the biosynthetic efficiency of cannabigerolic acid between synthetic GPP and reagent GPP was compared with cannabigerolic acid produced by reacting 0.9 gCDW/L cells, 1 mM olivetolic acid, 1 mM geranyl pyrophosphate lithium salt and 1 mM geranyl pyrophosphate tetrabutyl ammonium salt, and 5 mM magnesium chloride in 50 mM Tris-HCl buffer (pH8.0) for 24 hours. After obtaining 100 uL samples at set time intervals, cells were inactivated by adding 900 uL of methanol, filtered with a 0.2 μM PVDF filter (GE Healthcare, Pittsburgh, USA), and analyzed under the UPLC analysis conditions in Table 2.

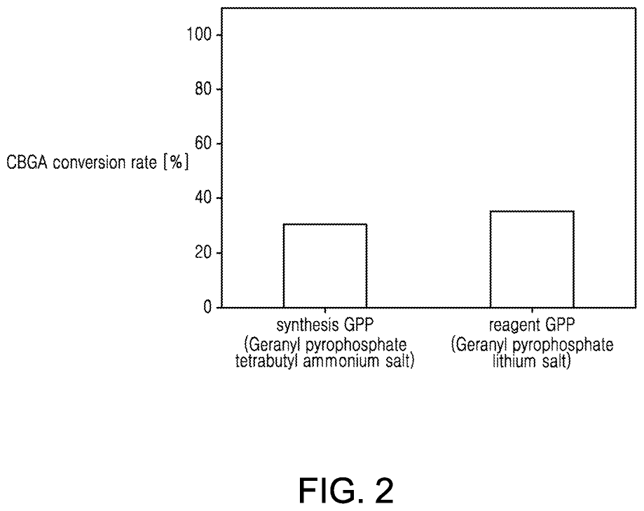

As a result of comparing the amount of CBGA produced between synthetic GPP and reagent GPP, it was confirmed that 0.30 mM of CBGA was produced in the case of synthetic GPP, similar to 0.35 mM of CBGA produced in the case of reagent GPP after 6 hours. This was a yield difference of approximately 5%, confirming that CBGA was successfully produced even with synthetic GPP (FIG. 2).

### 2.3. Optimization of Whole-Cell Reaction of S214T Mutant

### (1) Comparison of Cannabigerolic Acid Production (Conversion Rate) by Buffer pH

To each buffer of pH 3 to pH 10 (Table 4), 0.9 gCDW/L cells, 1 mM olivetolic acid and derivative thereof, 1.5 mM geranyl pyrophosphate tetrabutyl ammonium salt, and 5 mM magnesium chloride were added, and reacted for 6 hours to compare the produced cannabigerolic acid. After obtaining 100 uL samples at set time intervals, cells were inactivated by adding 900 uL of methanol, filtered with a 0.2 UM PVDF filter (GE Healthcare, Pittsburgh, USA), and analyzed under the UPLC analysis conditions in Table 3.

TABLE 4

| Buffer (100 mM) | pH |
|---|---|
| Sodium citrate | 3 |
| | 4 |
| | 6 |
| Potassium phosphate | 6 |
| | 7 |
| | 8 |
| Tris-HCl | 8 |
| | 10 |

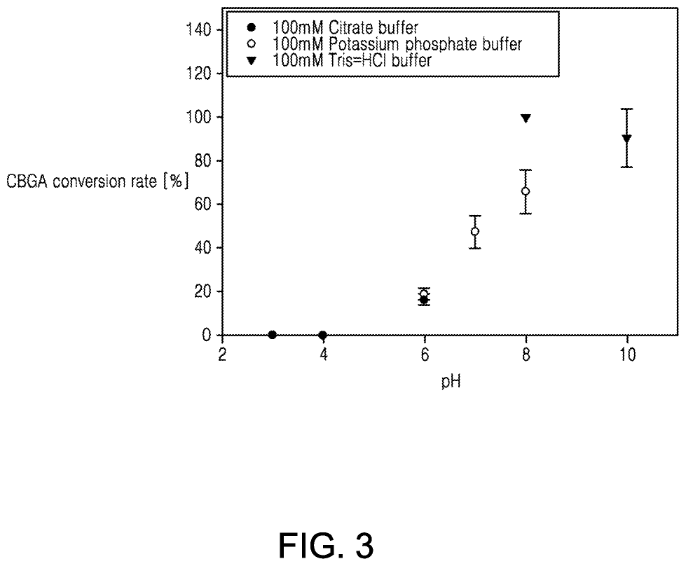

As a result of the whole-cell reaction, the whole-cell reaction conditions for the most active S214T mutant were optimized, and as a result of a comparison experiment of CBGA production by buffer pH using the S214T mutant, it was confirmed that Tris-HCl pH 8.0 showed the highest activity. Taking Tris-HCl pH 8.0 as the reference for 100% activity, the activity did not decrease significantly to 91% at pH 10, but the activity decreased to less than 50% at pH 7 and below, and no CBGA was produced from pH 4 (FIG. 3).

### (2) Comparison of Cannabigerolic Acid Production (Conversion Rate) by Concentration of Synthetic GPP

0.9 gCDW/L cells, 1 mM olivetolic acid and derivative thereof, 1.5 mM, 3 mM, 4.5 mM, or 6 mM synthetic GPP (geranyl pyrophosphate tetrabutyl ammonium salt), and 5 mM magnesium chloride were added in the buffer and reacted for 6 hours to compare the produced cannabigerolic acid. After obtaining 100 uL samples at set time intervals, cells were inactivated by adding 900 uL of methanol, filtered with a 0.2 μM PVDF filter (GE Healthcare, Pittsburgh, USA), and analyzed under the UPLC analysis conditions in Table 2.

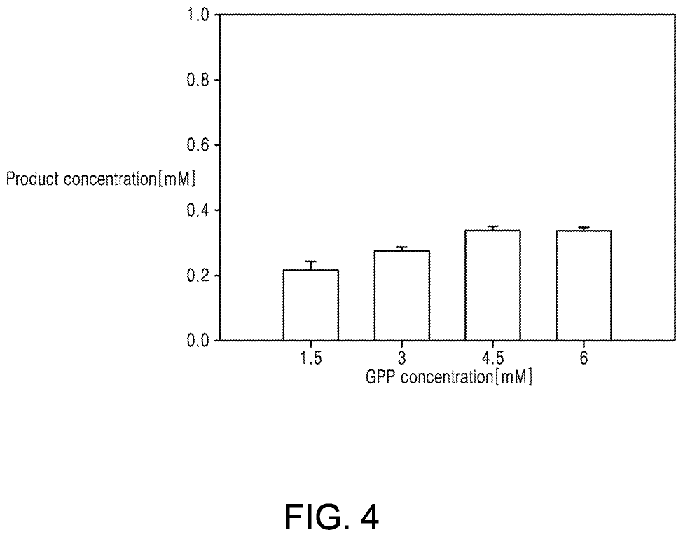

At Tris-HCl pH 8.0, which showed the highest activity, the amount of CBGA produced was compared depending on the concentration of GPP, the substrate, as a result, when the concentration of GPP was 4.5 mM or 6 mM, CBGA was produced at the same concentration of 0.34 mM (FIG. 4). Therefore, the optimal GPP concentration was selected as 4.5 mM.

### (3) Comparison of Cannabigerolic Acid Production (Conversion Rate) by Cell Concentration

Cannabigerolic acid produced by adding 0.9, 1.8, 3.6, 5.4, 7.2 gCDW/L cells, 1 mM olivetolic acid and its derivatives, 4.5 mM synthetic GPP (geranyl pyrophosphate tetrabutyl ammonium salt), and 5 mM magnesium chloride and reacting for 30 minutes was compared. After obtaining a 100 uL sample, 900 uL of methanol was added to inactivate the cells, which were filtered through a 0.2 μM PVDF filter (GE Healthcare, Pittsburgh, USA) and analyzed with the UPLC analysis conditions in Table 2.

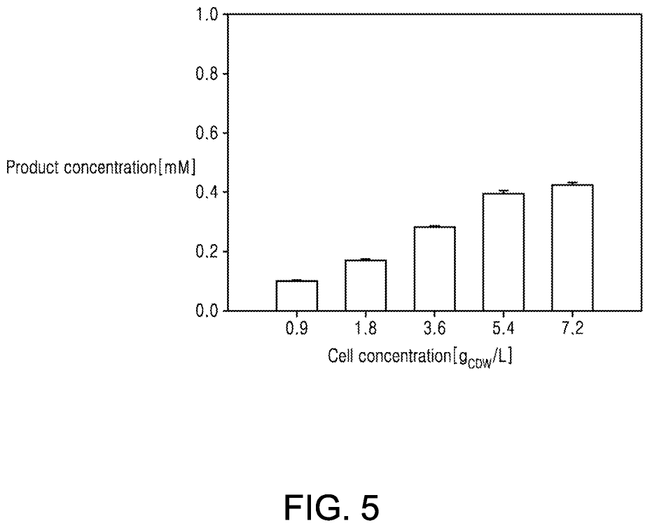

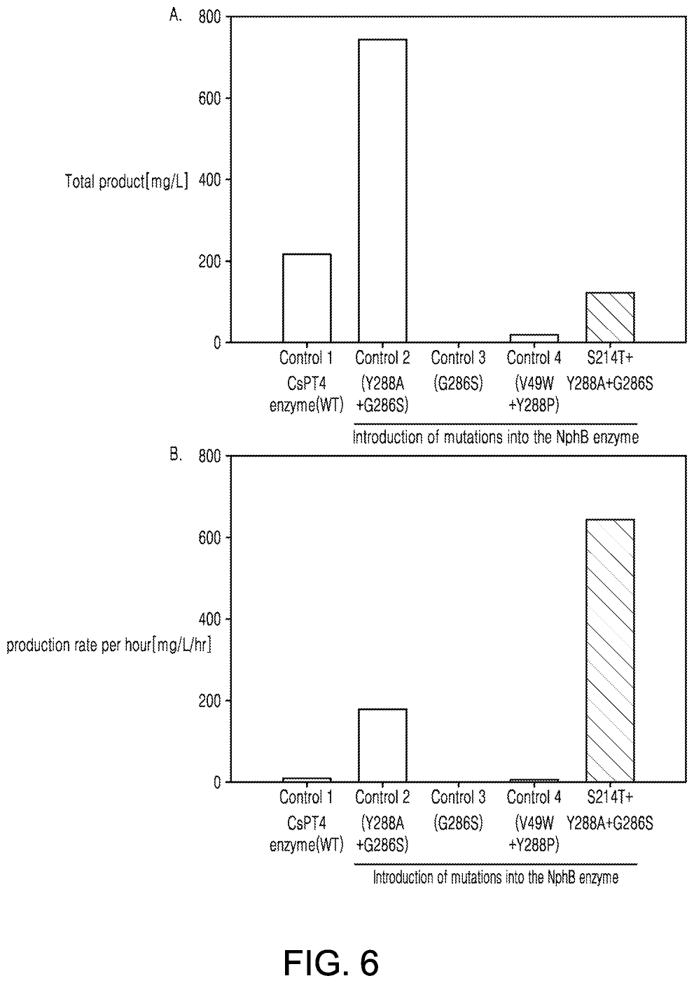

As a result, 0.10 mM of CBGA was produced at a cell concentration of 0.9 gCDW/L, 0.17 mM of CBGA at a cell concentration of 1.8 gCDW/L, 0.28 mM of CBGA at a cell concentration of 3.6 gCDW/L, CBGA 0.39 mM at a cell concentration of 5.4 gCDW/L, and CBGA 0.42 mM at a cell concentration of 7.2 gCDW/L (FIG. 5). Since similar concentrations of CBGA were produced at cell concentrations of 5.4 gCDW/L and 7.2 gCDW/L, the optimal cell concentration was chosen to be 5.4 gCDW/L. In conclusion, the optimal whole-cell reaction conditions for the S214T mutant were Tris-HCl PH 8.0, GPP 4.5 mM, and a cell concentration of 2.88 gCDW/L, and it was determined that under these conditions, the cannabigerolic acid of the disclosure showed a yield of about 40% in 30 minutes (hourly yield of more than 200 mg/L/hr), which was significantly higher than that of CsPT4 and other mutants (Y288A+G286S, G286S and V49W+Y288P), which may biosynthesize CBGA (FIG. 6).

### (4) Comparison of Cannabigerolic Acid Production (Conversion Rate) by Tag Type

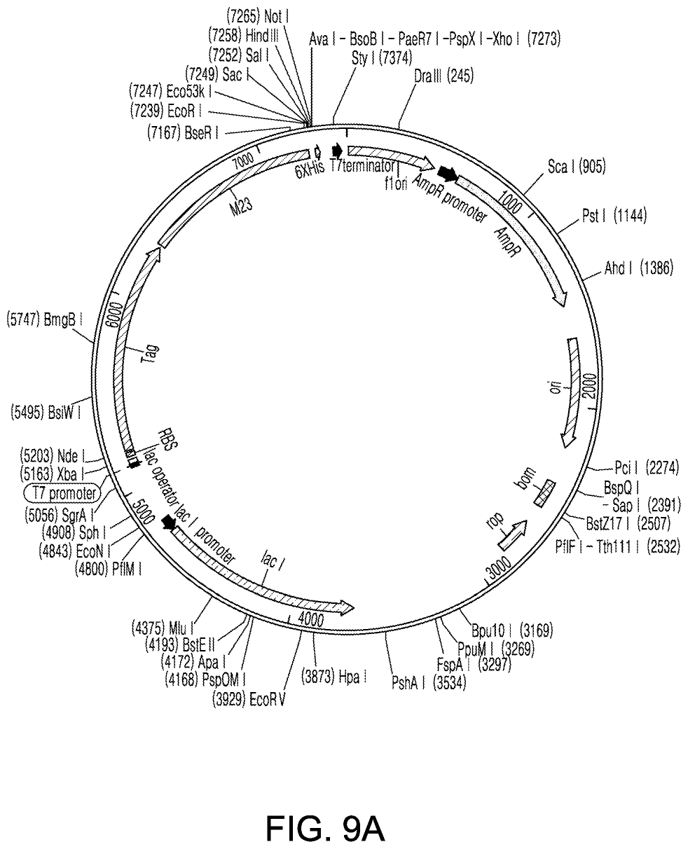

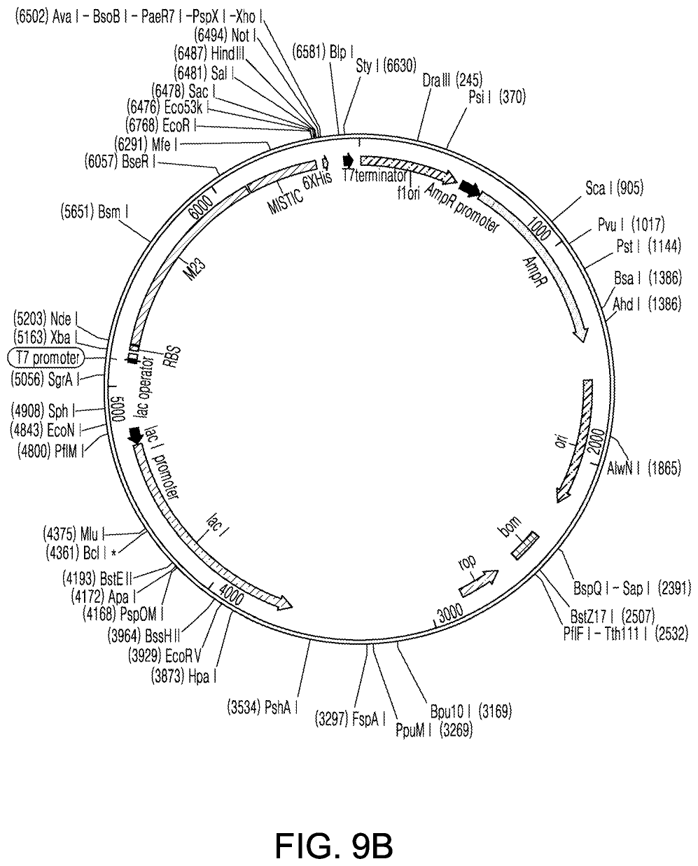

In order to compare the amount of cannabigerolic acid produced by tag type, the amount of cannabigerolic acid produced according to whole-cell reaction was compared using SUMO (nucleotide sequence: SEQ ID NO: 59, amino acid sequence: SEQ ID NO: 64), TrxA (nucleotide sequence: SEQ ID NO: 60, amino acid sequence: SEQ ID NO: 65), MBP (nucleotide sequence: SEQ ID NO: 61, amino acid sequence: SEQ ID NO: 66), Fh8 (nucleotide sequence: SEQ ID NO: 62, amino acid sequence: SEQ ID NO: 67), which are tags that may improve solubility to increase the probability of reacting with the substrate, and MISTIC (nucleotide sequence: SEQ ID NO: 63, amino acid sequence: SEQ ID NO: 68) tag, which may express enzymes on the cell surface (FIG. 9A). The MISTIC tag was attached to the N-terminus (MISTIC-N, FIG. 9A) or C-terminus (MISTIC-C, FIG. 9B) of the NphB enzyme to compare the amount of cannavirolic acid produced. For each tag, whole-cell reaction was performed with 0.9 gCDW/L of S214T mutant cells, and the reaction was performed for 6 hours in 50 mM Tris-HCl buffer (pH8.0) added with 1 mM olivetolic acid, 1 mM geranyl pyrophosphate lithium salt, 5 mM magnesium chloride (FhB, GST, SUMO, and Trx did not react, FIG. 8A). The nucleotide sequence (Table 5) and amino acid sequence (Table 6) of the tag used above are listed in the table below.

TABLE 5

| Sequence | | SEQ ID |
|---|---|---|
| classification | Nucleotide sequence | NO |
| SUMO | atgtccgacagcgaggtgaatcaggaagcgaagcctgaagttaagcc | 59 |
| | agaggtaaagcctgaaactcatataaatttgaaagtatcggacggttcg | |
| | agcgaaatattcttcaaaataaagaagactacgccgcttcgtagactgat | |
| | ggaggcattcgcaaagcggcagggcaaggagatggattctttacgcttt | |
| | ttgtatgacggcatacgtatccaggctgaccaaacgccggaagatttgg | |
| | atatggaggacaatgacattatcgaggcccaccgtgaacaaatcggc | |
| TrxA | atgtctgataaaatcattcatctgactgacgatagttttgatacggacgtac | 60 |
| | ttaaagcagatggagcaatcttagtagacttttgggccgagtggtgtggtc | |
| | catgcaaaatgatcgcccccatcttggatgaaattgccgacgaatacca | |
| | gggcaaactgacagtggcaaagctgaacattgaccaaaatcccggca | |
| | cagccccaaaatacgggattagaggcatacccacgctgttgctttttaag | |
| | aatggtgaagtcgcagctaccaaggtcggggctttaagcaagggaca | |
| | gttgaaggaatttctggatgccaatcttgcg | |
| MBP | atgaaaatcgaagaaggtaaactggtaatctggattaacggcgataaa | 61 |
| | ggctataacggtctcgctgaagtcggtaagaaattcgagaaagatacc | |
| | ggaattaaagtcaccgttgagcatccggataaactggaagagaaattc | |
| | ccacaggttgcggcaactggcgatggccctgacattatcttctgggcaca | |
| | cgaccgctttggtggctacgctcaatctggcctgttggctgaaatcacccc | |
| | ggacaaagcgttccaggacaagctgtatccgtttacctgggatgccgta | |
| | cgttacaacggcaagctgattgcttacccgatcgctgttgaagcgttatcg | |
| | ctgatttataacaaagatctgctgccgaacccgccaaaaacctgggaa | |
| | gagatcccggcgctggataaagaactgaaagcgaaaggtaagagcg | |
| | cgctgatgttcaacctgcaagaaccgtacttcacctggccgctgattgctg | |
| | ctgacgggggttatgcgttcaagtatgaaaacggcaagtacgacattaa | |
| | agacgtgggcgtggataacgctggcgcgaaagcgggtctgaccttcct | |
| | ggttgacctgattaaaaacaaacacatgaatgcagacaccgattactcc | |
| | atcgcagaagctgcctttaataaaggcgaaacagcgatgaccatcaac | |
| | ggcccgtgggcatggtccaacatcgacaccagcaaagtgaattatggt | |
| | gtaacggtactgccgaccttcaagggtcaaccatccaaaccgttcgttgg | |
| | cgtgctgagcgcaggtattaacgccgccagtccgaacaaagagctgg | |
| | caaaagagttcctcgaaaactatctgctgactgatgaaggtctggaagc | |
| | ggttaataaagacaaaccgctgggtgccgtagcgctgaagtcttacgag | |
| | gaagagttggtgaaagatccgcgtattgccgccactatggaaaacgcc | |
| | cagaaaggtgaaatcatgccgaacatcccgcagatgtccgctttctggt | |
| | atgccgtgcgtactgcggtgatcaacgccgccagcggtcgtcagactgt | |
| | cgatgaagccctgaaagacgcgcagact | |
| Fh8 | atgccaagcgtgcaagaagttgaaaaattacttcacgttcttgatcggaa | 62 |
| | tggggatgggaaagtgagtgccgaggaattgaaagcatttgcagatga | |
| | cagtaaatgtcccttggacagtaacaaaattaaggcgtttattaaggaac | |
| | atgataaaaacaaggatggcaaattggaccttaaagaattagtaagcat | |
| | cttgagttct | |
| MISTIC | atgttttgtacttttttcgagaagcaccaccggaagtgggatatattgttaga | 63 |
| | gaaaagcaccggggtcatggaagcaatgaaggttacatcagaggaa | |
| | aaagaacaactttccacagccatagaccgcatgaatgagggtcttgatg | |
| | ctttcatccaattgtataacgagtccgaaatagacgagccacttatccag | |
| | cttgacgatgacacagcggaattgatgaaacaagcccgcgacatgtat | |
| | ggacaggagaagttgaatgagaaattaaacacaatcatcaagcagat | |
| | attgagcatttctgtgagcgaagaaggcgagaaggaatga | |

TABLE 6

| Sequence | | SEQ ID |
|---|---|---|
| classification | Amino acid sequence | NO |
| SUMO | MSDSEVNQEAKPEVKPEVKPETHINLKVSDGSSEIF | 64 |
| | FKIKKTTPLRRLMEAFAKRQGKEMDSLRFLYDGIRI | |
| | QADQTPEDLDMEDNDIIEAHREQIG | |
| TrxA | MSDKIIHLTDDSFDTDVLKADGAILVDFWAEWCGPC | 65 |
| | KMIAPILDEIADEYQGKLTVAKLNIDQNPGTAPKYGI | |
| | RGIPTLLLFKNGEVAATKVGALSKGQLKEFLDANLA | |
| MBP | MKIEEGKLVIWINGDKGYNGLAEVGKKFEKDTGIKV | 66 |
| | TVEHPDKLEEKFPQVAATGDGPDIIFWAHDRFGGY | |
| | AQSGLLAEITPDKAFQDKLYPFTWDAVRYNGKLIAY | |
| | PIAVEALSLIYNKDLLPNPPKTWEEIPALDKELKAKG | |
| | KSALMFNLQEPYFTWPLIAADGGYAFKYENGKYDIK | |
| | DVGVDNAGAKAGLTFLVDLIKNKHMNADTDYSIAEA | |
| | AFNKGETAMTINGPWAWSNIDTSKVNYGVTVLPTF | |
| | KGQPSKPFVGVLSAGINAASPNKELAKEFLENYLLT | |
| | DEGLEAVNKDKPLGAVALKSYEEELVKDPRIAATME | |
| | NAQKGEIMPNIPQMSAFWYAVRTAVINAASGRQTV | |
| | DEALKDAQT | |
| Fh8 | MPSVQEVEKLLHVLDRNGDGKVSAEELKAFADDSK | 67 |
| | CPLDSNKIKAFIKEHDKNKDGKLDLKELVSILSS | |
| MISTIC | MFCTFFEKHHRKWDILLEKSTGVMEAMKVTSEEKE | 68 |
| | QLSTAIDRMNEGLDAFIQLYNESEIDEPLIQLDDDTA | |
| | ELMKQARDMYGQEKLNEKLNTIIKQILSISVSEEGEK | |
| | E | |

As a result of an experiment comparing the amount of CBGA produced by tag type using the S214T mutant, it was confirmed that FhB, GST, SUMO, and Trx tags almost did not elicit a response, while MBP and MISTIC-C tags showed a significant increase in CBGA production by 3-fold and 6-fold, respectively, compared to the conditions without these tags (FIG. 8A). Likewise, the consumption rate of olivetolic acid, which was used as a substrate for CBGA synthesis, was also confirmed to be fastest when MBP and MISTIC-C tags were used (FIG. 8B).

From the above description, those skilled in the art to which the disclosure pertains will be able to understand that the disclosure may be implemented in other specific forms without changing its technical idea or essential features. In this regard, it should be understood that the embodiments described above are for example in all respects and are not intended to be limiting. The scope of the disclosure is to be construed to include the meaning and scope of the patent claims hereinafter set forth, and all modifications or variations derived from the equivalents thereof, rather than the detailed description above.

The disclosure may provide polypeptides including prenyltransferase variants, polynucleotides encoding them, recombinant vectors including the polynucleotides, recombinant microorganisms for the production of cannabigerolic acid and derivative thereof, and method of producing cannabigerolic acid and derivative thereof.

The recombinant microorganism for producing cannabigerolic acid and derivative thereof transformed with the recombinant vector of the disclosure has increased biosynthetic reactivity, and may stably mass-produce cannabigerolic acid and derivative thereof.

The method of producing cannabigerolic acid and derivative thereof of the disclosure may produce various cannabinoids with high productivity and yield at a low production cost.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. A polypeptide comprising a prenyltransferase variant, wherein the prenyltransferase variant is a polypeptide comprising one or more amino acid substitutions in an amino acid sequence of SEQ ID NO: 2.

2. The polypeptide of claim 1,
wherein the one or more amino acid substitutions comprise one or more amino acid substitutions selected from the group consisting of 47th, 49th, 213rd, 214th, 216th, 232nd, 269th, 271st, 286th and 288th positions of SEQ ID NO: 2.

3. The polypeptide of claim 1,
wherein the amino acid substitutions comprise one or more substitutions selected from the group consisting of: a substitution of phenylalanine at the 213th position of SEQ ID NO: 2 with tyrosine; and a substitution of serine at the 214th position of SEQ ID NO: 2 with threonine.

4. The polypeptide of claim 1,
wherein the amino acid substitutions comprise a substitution of phenylalanine at the 213rd position of SEQ ID NO: 2 with tyrosine, a substitution of glycine at the 286th position of SEQ ID NO: 2 with serine, and a substitution of tyrosine at the 288th position of SEQ ID NO: 2 with alanine.

5. The polypeptide of claim 1,
wherein the amino acid substitutions comprise a substitution of serine at the 214th position of SEQ ID NO: 2 with threonine, a substitution of glycine at the 286th position of SEQ ID NO: 2 with serine, and a substitution of tyrosine at the 288th position of SEQ ID NO: 2 with alanine.

6. The polypeptide of claim 1,
further comprising an affinity tag.

7. The polypeptide of claim 6,
wherein the affinity tag is a maltose binding protein (MBP) tag or a MISTIC tag.

8. The polypeptide of claim 7,
wherein the MISTIC tag is linked to a C-terminus of the prenyltransferase variant.

9. A polynucleotide encoding the polypeptide of claim 1.

10. A recombinant vector comprising the polynucleotide of claim 9.

11. A recombinant microorganism for production of cannabigerolic acid and derivatives thereof, transformed with the vector of claim 10.

12. The recombinant microorganism of claim 11,
wherein the derivatives of cannabigerolic acid are at least one selected from the group consisting of cannabigerorcinic acid, cannabigerovarinic acid, and 3-geranyl-2,4-hydroxybenzoic acid.

13. The recombinant microorganism of claim 11,
wherein the recombinant microorganism has increased cannabigerolic acid biosynthetic reactivity.

14. A method of producing cannabigerolic acid or derivatives thereof, comprising a process of culturing, in a solution comprising geranyl pyrophosphate and either olivetolic acid or a derivative thereof, a microorganism expressing a polypeptide comprising a prenyltransferase variant,
wherein the prenyltransferase variant is a polypeptide comprising one or more amino acid substitutions in an amino acid sequence of SEQ ID NO: 2.

15. The method of claim 14,
wherein the geranyl pyrophosphate is geranyl pyrophosphate tetrabutyl ammonium salt.

16. The method of claim 14,
wherein the derivatives of cannabigerolic acid are at least one selected from the group consisting of cannabigerorcinic acid, cannabigerovarinic acid, and 3-geranyl-2,4-hydroxybenzoic acid.

17. The method of claim 14,
wherein the microorganism is a microorganism transformed with a recombinant vector comprising a polynucleotide encoding the polypeptide comprising the prenyltransferase variant.

18. (canceled)


SEQUENCE LISTING (68 sequences)

SEQ ID NO: 1 - RNA, 924 nt, Streptomyces sp.
  source 1..924: mol_type = mRNA
atgagcgaagcagctgatgtcgaaagagtgtacgctgcgatggaagaggccgccggtcttttaggagtcgcttgcgccagagataagatctaccctctgttatctactttccaggacacattagtagaggggggatctgttgtggtattctcgatggctagtggtcgccattcaactgaactggatttctctataagtgtacctacgagtcatggcgatccctatgccacggtagttgaaaaaggattatttccagctacaggccatcccgttgatgacttactggctgatacacagaaacacttgccagtgagtatgtttgctatcgacggagaggttacaggcggatttaagaagacgtatgcctttttcccaacggacaacatgcctggcgtggctgaactttcagctattccttcgatgccaccggccgtggccgagaatgcagaattgttcgctcgttatggcttagacaaggtacaaatgaccagcatggattacaaaaagagacaagttaatctttatttctctgaacttagcgctcaaacgttagaagctgagagtgtattagctttagtgcgggagcttggcctgcacgttccaaacgaactgggattgaaattttgtaagcgttcattcagtgtatatcctactcttaactgggaaacaggcaaaatcgaccggctttgctttgcagttatctcaaatgatccaactcttgtgccttcatctgatgaaggcgacatagagaaattccataattatgccacaaaggcaccttatgcgtatgtcggagagaaacgcacccttgtttacggtttgaccttatccccgaaagaggagtattacaaattaggtgcttattatcatataacagatgtgcaaagagggttgcttaaagccttcgacagccttgaagactga

SEQ ID NO: 2 - protein, 307 aa, Streptomyces sp. [sequence omitted]

SEQ ID NO: 3 - DNA, 27 bp, synthetic construct
  source 1..27: mol_type = other DNA
ggatctcttgtggtattctcgatggct

SEQ ID NO: 4 - DNA, 27 bp, synthetic construct
  source 1..27: mol_type = other DNA
taccacaagagatcccccctctactaa

SEQ ID NO: 5 - DNA, 27 bp, synthetic construct
  source 1..27: mol_type = other DNA
ggatctatggtggtattctcgatggct

SEQ ID NO: 6 - DNA, 27 bp, synthetic construct
  source 1..27: mol_type = other DNA
taccaccatagatcccccctctactaa

SEQ ID NO: 7 - DNA, 27 bp, synthetic construct
  source 1..27: mol_type = other DNA
ggatctattgtggtattctcgatggct

SEQ ID NO: 8 - DNA, 27 bp, synthetic construct
  source 1..27: mol_type = other DNA
taccacaatagatcccccctctactaa

SEQ ID NO: 9 - DNA, 27 bp, synthetic construct
  source 1..27: mol_type = other DNA
ggatctgctgtggtattctcgatggct

SEQ ID NO: 10 - DNA, 27 bp, synthetic construct
  source 1..27: mol_type = other DNA
taccacagcagatcccccctctactaa

SEQ ID NO: 11 - DNA, 27 bp, synthetic construct
  source 1..27: mol_type = other DNA
ggatctaaagtggtattctcgatggct

SEQ ID NO: 12 - DNA, 27 bp, synthetic construct
  source 1..27: mol_type = other DNA
taccactttagatcccccctctactaa

SEQ ID NO: 13 - DNA, 27 bp, synthetic construct
  source 1..27: mol_type = other DNA
ggatctcgtgtggtattctcgatggct

SEQ ID NO: 14 - DNA, 27 bp, synthetic construct
  source 1..27: mol_type = other DNA
taccacacgagatcccccctctactaa

SEQ ID NO: 15 - DNA, 27 bp, synthetic construct
  source 1..27: mol_type = other DNA
gttgtgctattctcgatggctagtggt

SEQ ID NO: 16 - DNA, 27 bp, synthetic construct
  source 1..27: mol_type = other DNA
cgagaatagcacaacagatcccccctc

SEQ ID NO: 17 - DNA, 27 bp, synthetic construct
  source 1..27: mol_type = other DNA
gttgtgatgttctcgatggctagtggt

SEQ ID NO: 18 - DNA, 27 bp, synthetic construct
  source 1..27: mol_type = other DNA
cgagaacatcacaacagatcccccctc

SEQ ID NO: 19 - DNA, 27 bp, synthetic construct
  source 1..27: mol_type = other DNA
gttgtgatattctcgatggctagtggt

SEQ ID NO: 20 - DNA, 27 bp, synthetic construct
  source 1..27: mol_type = other DNA
cgagaatatcacaacagatcccccctc

SEQ ID NO: 21 - DNA, 27 bp, synthetic construct
  source 1..27: mol_type = other DNA
gttgtggcattctcgatggctagtggt

SEQ ID NO: 22 - DNA, 27 bp, synthetic construct
  source 1..27: mol_type = other DNA
cgagaatgccacaacagatcccccctc

SEQ ID NO: 23 - DNA, 27 bp, synthetic construct
  source 1..27: mol_type = other DNA
gttgtgaaattctcgatggctagtggt

SEQ ID NO: 24 - DNA, 27 bp, synthetic construct
  source 1..27: mol_type = other DNA
cgagaatttcacaacagatcccccctc

SEQ ID NO: 25 - DNA, 27 bp, synthetic construct
  source 1..27: mol_type = other DNA
gttgtgcgattctcgatggctagtggt

SEQ ID NO: 26 - DNA, 27 bp, synthetic construct
  source 1..27: mol_type = other DNA
cgagaatcgcacaacagatcccccctc

SEQ ID NO: 27 - DNA, 27 bp, synthetic construct
  source 1..27: mol_type = other DNA
cgttcatacagtgtatatcctactctt

SEQ ID NO: 28 - DNA, 27 bp, synthetic construct
  source 1..27: mol_type = other DNA
tacactgtatgaacgcttacaaaattt

SEQ ID NO: 29 - DNA, 27 bp, synthetic construct
  source 1..27: mol_type = other DNA
tcattcactgtatatcctactcttaac

SEQ ID NO: 30 - DNA, 27 bp, synthetic construct
  source 1..27: mol_type = other DNA
atatacagtgaatgaacgcttacaaaa

SEQ ID NO: 31 - DNA, 27 bp, synthetic construct
  source 1..27: mol_type = other DNA
tcattcgctgtatatcctactcttaac

SEQ ID NO: 32 - DNA, 27 bp, synthetic construct
  source 1..27: mol_type = other DNA
atatacagcgaatgaacgcttacaaaa

SEQ ID NO: 33 - DNA, 27 bp, synthetic construct
  source 1..27: mol_type = other DNA
agtgtatttcctactcttaactgggaa

SEQ ID NO: 34 - DNA, 27 bp, synthetic construct
  source 1..27: mol_type = other DNA
agtaggaaatacactgaatgaacgctt

SEQ ID NO: 35 - DNA, 27 bp, synthetic construct
  source 1..27: mol_type = other DNA
agtgtaatgcctactcttaactgggaa

SEQ ID NO: 36 - DNA, 27 bp, synthetic construct
  source 1..27: mol_type = other DNA
agtaggcattacactgaatgaacgctt

SEQ ID NO: 37 - DNA, 27 bp, synthetic construct
  source 1..27: mol_type = other DNA
agtgtacttcctactcttaactgggaa

SEQ ID NO: 38 - DNA, 27 bp, synthetic construct
  source 1..27: mol_type = other DNA
agtaggaagtacactgaatgaacgctt

SEQ ID NO: 39 - DNA, 27 bp, synthetic construct
  source 1..27: mol_type = other DNA
agtgtaattcctactcttaactgggaa

SEQ ID NO: 40 - DNA, 27 bp, synthetic construct
  source 1..27: mol_type = other DNA
agtaggaattacactgaatgaacgctt

SEQ ID NO: 41 - DNA, 27 bp, synthetic construct
  source 1..27: mol_type = other DNA
tgctttatagttatctcaaatgatcca

SEQ ID NO: 42 - DNA, 27 bp, synthetic construct
  source 1..27: mol_type = other DNA
gataactataaagcaaagccggtcgat

SEQ ID NO: 43 - DNA, 27 bp, synthetic construct
  source 1..27: mol_type = other DNA
tgctttttagttatctcaaatgatcca

SEQ ID NO: 44 - DNA, 27 bp, synthetic construct
  source 1..27: mol_type = other DNA
gataactaaaaagcaaagccggtcgat

SEQ ID NO: 45 - DNA, 27 bp, synthetic construct
  source 1..27: mol_type = other DNA
tgctttatggttatctcaaatgatcca

SEQ ID NO: 46 - DNA, 27 bp, synthetic construct
  source 1..27: mol_type = other DNA
gataaccataaagcaaagccggtcgat

SEQ ID NO: 47 - DNA, 27 bp, synthetic construct
  source 1..27: mol_type = other DNA
tgctttgtagttatctcaaatgatcca

SEQ ID NO: 48 - DNA, 27 bp, synthetic construct
  source 1..27: mol_type = other DNA
gataactacaaagcaaagccggtcgat

SEQ ID NO: 49 - DNA, 27 bp, synthetic construct
  source 1..27: mol_type = other DNA
aaacgcatgcttgtttacggtttgacc

SEQ ID NO: 50 - DNA, 27 bp, synthetic construct
  source 1..27: mol_type = other DNA
aacaagcatgcgtttctctccgacata

SEQ ID NO: 51 - DNA, 27 bp, synthetic construct
  source 1..27: mol_type = other DNA
aaacgcatccttgtttacggtttgacc

SEQ ID NO: 52 - DNA, 27 bp, synthetic construct
  source 1..27: mol_type = other DNA
aacaaggatgcgtttctctccgacata

SEQ ID NO: 53 - DNA, 27 bp, synthetic construct
  source 1..27: mol_type = other DNA
aaacgcctccttgtttacggtttgacc

SEQ ID NO: 54 - DNA, 27 bp, synthetic construct
  source 1..27: mol_type = other DNA
aacaaggaggcgtttctctccgacata

SEQ ID NO: 55 - DNA, 27 bp, synthetic construct
  source 1..27: mol_type = other DNA
aaacgcgtccttgtttacggtttgacc

SEQ ID NO: 56 - DNA, 27 bp, synthetic construct
  source 1..27: mol_type = other DNA
aacaaggacgcgtttctctccgacata

SEQ ID NO: 57 - DNA, 27 bp, synthetic construct
  source 1..27: mol_type = other DNA
acccttttttacggtttgaccttatcc

SEQ ID NO: 58 - DNA, 27 bp, synthetic construct
  source 1..27: mol_type = other DNA
accgtaaaaaagggtgcgtttctctcc

SEQ ID NO: 59 - RNA, 291 nt, synthetic construct
  source 1..291: mol_type = other RNA
atgtccgacagcgaggtgaatcaggaagcgaagcctgaagttaagccagaggtaaagcctgaaactcatataaatttgaaagtatcggacggttcgagcgaaatattcttcaaaataaagaagactacgccgcttcgtagactgatggaggcattcgcaaagcggcagggcaaggagatggattctttacgctttttgtatgacggcatacgtatccaggctgaccaaacgccggaagatttggatatggaggacaatgacattatcgaggcccaccgtgaacaaatcggc

SEQ ID NO: 60 - RNA, 327 nt, synthetic construct
  source 1..327: mol_type = other RNA
atgtctgataaaatcattcatctgactgacgatagttttgatacggacgtacttaaagcagatggagcaatcttagtagacttttgggccgagtggtgtggtccatgcaaaatgatcgcccccatcttggatgaaattgccgacgaataccagggcaaactgacagtggcaaagctgaacattgaccaaaatcccggcacagccccaaaatacgggattagaggcatacccacgctgttgctttttaagaatggtgaagtcgcagctaccaaggtcggggctttaagcaagggacagttgaaggaatttctggatgccaatcttgcg

SEQ ID NO: 61 - RNA, 1101 nt, synthetic construct
  source 1..1101: mol_type = other RNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagact

SEQ ID NO: 62 - RNA, 207 nt, synthetic construct
  source 1..207: mol_type = other RNA
atgccaagcgtgcaagaagttgaaaaattacttcacgttcttgatcggaatggggatgggaaagtgagtgccgaggaattgaaagcatttgcagatgacagtaaatgtcccttggacagtaacaaaattaaggcgtttattaaggaacatgataaaaacaaggatggcaaattggaccttaaagaattagtaagcatcttgagttct

SEQ ID NO: 63 - RNA, 333 nt, synthetic construct
  source 1..333: mol_type = other RNA
atgttttgtacttttttcgagaagcaccaccggaagtgggatatattgttagagaaaagcaccggggtcatggaagcaatgaaggttacatcagaggaaaaagaacaactttccacagccatagaccgcatgaatgagggtcttgatgctttcatccaattgtataacgagtccgaaatagacgagccacttatccagcttgacgatgacacagcggaattgatgaaacaagcccgcgacatgtatggacaggagaagttgaatgagaaattaaacacaatcatcaagcagatattgagcatttctgtgagcgaagaaggcgagaaggaatga

SEQ ID NO: 64 - protein, 97 aa, synthetic construct
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIG

SEQ ID NO: 65 - protein, 109 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 66 - protein, 367 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQT

SEQ ID NO: 67 - protein, 69 aa, synthetic construct
MPSVQEVEKLLHVLDRNGDGKVSAEELKAFADDSKCPLDSNKIKAFIKEHDKNKDGKLDLKELVSILSS

SEQ ID NO: 68 - protein, 110 aa, synthetic construct
MFCTFFEKHHRKWDILLEKSTGVMEAMKVTSEEKEQLSTAIDRMNEGLDAFIQLYNESEIDEPLIQLDDDTAELMKQARDMYGQEKLNEKLNTIIKQILSISVSEEGEKE